(19) United States
(12) Patent Application Publication
(10) Pub. No.: US 2010/0310629 A1
(43) Pub. Date: Dec. 9, 2010

(54) INTRAPERICARDIAL ANTIARRHYTHMIC DELIVERY

(76) Inventor: Richard J. GRAY, Carmel Valley, CA (US)

Correspondence Address: MCDERMOTT WILL & EMERY LLP, 18191 VON KARMAN AVE., SUITE 500, IRVINE, CA, 92612-7108, US

(21) Appl. No.: 12/645,423

(22) Filed: Dec. 22, 2009

Related U.S. Application Data
(60) Provisional application No. 61/140,595, filed on Dec. 23, 2008.

(51) Int. Cl. A61F 2/00; A61K 31/34

(52) U.S. Cl. 424/426; 514/469

(57) ABSTRACT

Certain embodiments provide a method, of treating or reducing a risk of postoperative cardiac arrhythmia, including: creating an opening in a mammal's body; advancing a releasing member from outside the body, through the opening, and toward the mammal's heart; positioning the releasing member between a visceral layer and a parietal layer of pericardium of the mammal's heart; and at least partially closing the opening in the body, leaving the releasing member in the pericardial space. In certain embodiments, the releasing member releases an antiarrhythmic drug into the mammal's pericardial space and is configured not to impede significantly a systolic or a diastolic function of the heart while the releasing member resides in the pericardial space.

### RELATED APPLICATIONS

This application claims the benefit of U.S. Provisional Application Ser. No. 61/140,595, filed on Dec. 23, 2008, and titled “Intrapericardial Antiarrhythmic Delivery”, the entirety of which is hereby incorporated by reference.

### FIELD

Embodiments of the subject disclosure relate to imaging of cardiac tissues and atrial fibrillation treatments.

### BACKGROUND

Atrial fibrillation (AF) is a cardiac disorder involving an irregular, and often ineffective, quiver-type beating of the heart's two upper chambers (the atria). In certain forms of AF, blood may not be pumped completely out of the atria, pool along the atrial walls, and eventually clot. If a blood clot in the atria leaves the heart and becomes lodged in a brain artery, stroke can result.

AF affects more than 2.2 million people in the United States, and the prevalence of AF increases with age. Approximately 4% of people over age 60 have experienced an episode of AF. AF can occur in healthy people, but more often is associated with an underlying condition such as coronary heart disease, hypertension, valvular heart disease, rheumatic heart disease cardiac surgery, or pulmonary surgery.

Treatments for AF include medications to decrease blood clotting, medications to slow down rapid heart rate, electric shock to restore normal heart rhythm (cardioversion), pulmonary vein antrum isolation (PVAI), and use of pacemakers to regulate heart rhythm.

Normally, a mammalian heart beat comprises phases called “diastole,” in which the heart relaxes and fills with blood, and “systole,” in which the heart contracts and pumps out the blood. An electrical wavefront typically starts in the “sinoatrial” (SA) node of the atrium, spreads over the two atria, and leads to contraction of cardiac muscle. When such an electrical wavefront reaches the “atrioventricular” (AV) node, the wavefront is delayed, allowing the atria to finish contracting, moving blood from the atria to the ventricles.

From the AV node, the electrical wavefront spreads through the His-Purkinje system, which comprises fibers that form a specialized conduction system that quickly propagates the wavefront throughout the ventricles, resulting in ventricular contraction. Contraction of the ventricles pumps blood into the lungs and body. At the end of contracting, the ventricles relax and the process repeats.

An electrocardiogram (ECG) can be used to assess heart rhythm and disturbances therein by measuring electrical activities of the heart that are detectable at surfaces of the body. An ECG typically comprises a repeated pattern of three measured electrical waveform components of a heartbeat: the “P wave,” the “Q wave,” and the “T wave.” The P wave results from atrial depolarization, i.e., the wavefront generated as electrical impulses from the SA node spread throughout the atrial musculature. The Q wave occurs at the beginning of a “QRS complex,” but may not always be present. The T wave involves electrical recovery of the ventricles.

The P wave precedes the QRS complex, which occurs as a result of ventricular depolarization. The QRS complex, a large waveform, typically comprises three waves, the “Q wave,” the “R wave,” and the “S wave,” but not every QRS complex contains a Q wave, an R wave, and an S wave. By convention, any combination of these waves can be referred to as a QRS complex. The Q wave represents depolarization of the interventricular septum. The R wave is typically the first positive deflection, and the S wave is the negative deflection that follows the R wave. The time interval between two consecutive beats, the so-called “beat interval,” is often measured from the R-wave of one beat to the R-wave of the following beat, and the time between two consecutive R waves is called the RR interval. A “PR interval” comprises the time it takes an electrical impulse to travel from the atria through the AV node, bundle of His, and bundle branches to the Purkinje's fibers; and the PR interval extends from the beginning of the P wave to the beginning of the QRS complex.

The QRS complex is usually the dominant feature of an ECG. The P wave is much smaller than the QRS complex because the atria generate less electrical activity than the larger ventricles. Other components of an ECG include the “Q-T Interval,” which represents the time necessary for ventricular depolarization and repolarization, and extends from the beginning of the QRS complex to the end of a T wave. By analyzing patterns of an ECG, insights into the condition of the heart can be obtained.

In an ECG from a heart with normal rhythm, large QRS complexes are separated by a fairly flat signal, except for a small upright bump (the P wave) about 120-200 ms before the QRS complex. A P wave is conducted when atrial electrical activity conducts through the AV node, causing electrical activation of the ventricles and the QRS complex. At most one P wave in an RR interval is conducted, and any other P waves in the same RR interval are non-conducted. A P wave is non-conducted when it fails to lead to a QRS complex. Non-conducted P waves can result from a premature P wave, a condition called AV block, and other reasons. P waves non-conducted as a result of AV block are said to be blocked P waves.

In atrial flutter, the atrial rhythm can increase to approximately 250-350 beats per minute. Increased atrial rhythms are sometimes detected as continuous waves in an ECG, with several waves appearing in a continuous, connected pattern in each RR interval: a pattern substantially different from the normal pattern of a single P wave in each RR interval. Such waves of continuous, cyclic atrial activity are called flutter waves or F-waves, and may form a sawtooth pattern in an ECG. During atrial flutter, the ventricular response can become locked into a regular pattern with the atrial activity, so that, for instance, every third flutter wave results in a QRS complex while the other flutter waves are non-conducted. In other cases, conduction of the flutter waves can be more random, resulting in an irregular ventricular rhythm.

Rapid atrial rhythm rates, generally over 350-400 beats per minute, are called AF. Such atrial activity can be visible in the RR interval as continuous, cyclic activity referred to as “f waves,” or coarse AF. Typically, the f waves are cyclic, but not as organized or consistent in shape as the F waves of atrial flutter. When viewed in two ECG channels, the cyclic activity of f waves may be seen to alternate back and forth between channels in what appears to be modulated electrical activity. At other times, AF may be present with no obvious cyclic activity visible in an ECG, but with low amplitude disorganized “noise” in the baseline. In other cases, there may be total absence of atrial activity, suggesting that the AF has become disorganized.

### SUMMARY

Certain embodiments provide a method, of treating or reducing a risk of postoperative cardiac arrhythmia, comprising: creating an opening in a mammal's body; advancing a releasing member from outside the body, through the opening, and toward the mammal's heart; positioning the releasing member between a visceral layer and a parietal layer of pericardium of the mammal's heart; and at least partially closing the opening in the body, leaving the releasing member in the pericardial space. In certain embodiments, the releasing member releases an antiarrhythmic drug into the mammal's pericardial space and is configured not to impede significantly a systolic or a diastolic function of the heart while the releasing member resides in the pericardial space.

In certain embodiments, the positioning occurs before or after a cardiopulmonary surgery on the heart that is performed before the at least partial closing.

In certain embodiments, the cardiopulmonary procedure comprises at least one surgery selected from surgery to bypass or repair stenotic coronary arteries; surgery for a heart valve; surgery to correct a cardiac arrhythmia; surgery to remove heart muscle tissue; heart transplant surgery; lung surgery; and surgery to correct a congenital heart disease.

In certain embodiments, the antiarrhythmic drug comprises at least one of quinidine, procainamide, disopyramide, lidocaine, phenytoin, mexiletine, flecainide, propafenone, moricizine, propranolol, esmolol, timolol, metoprolol, atenolol, amiodarone, sotalol, ibutilide, dofetilide, verapamil, diltiazem, adenosine, digoxin, amiodarone, dofetilide, sotalol droperidol, levomethadyl, spafloxacin, thioridazine, cisapride.

In certain embodiments, the arrhythmia comprises atrial fibrillation. In certain embodiments, the arrhythmia comprises atrial fibrillation, and the antiarrhythmic drug comprises amiodarone. In certain embodiments, the releasing member comprises a biodegradable material. In certain embodiments, the releasing member releases the antiarrhythmic drug for a period of from about one day to about four days after the positioning.

In certain embodiments, a method comprises permitting at least a portion of the releasing member to biodegrade in the mammal's body.

Certain embodiments provide a device, for treating or reducing a risk of postsurgical cardiac arrhythmia, comprising: a releasing member configured to be placed from outside a mammal's body, through an opening in the body, and into the mammal, and configured to be positioned between a visceral layer and a parietal layer of pericardium of the mammal's heart. In certain embodiments, the releasing member (i) releases an antiarrhythmic drug into the mammal's pericardial space, and (ii) is configured not to impede significantly a systolic or a diastolic function of the heart while the releasing member resides in the pericardial space. In certain embodiments, the releasing member at least partially resorbs in the mammal's body over at least two days following placement in the pericardial space. In certain embodiments, the device further comprises the antiarrhythmic drug. In some embodiments, the antiarrhythmic drug comprises amiodarone.

In certain embodiments, the antiarrhythmic drug comprises at least one of quinidine, procainamide, disopyramide, lidocaine, phenytoin, mexiletine, flecainide, propafenone, moricizine, propranolol, esmolol, timolol, metoprolol, atenolol, amiodarone, sotalol, ibutilide, dofetilide, verapamil, diltiazem, adenosine, digoxin, amiodarone, dofetilide, sotalol droperidol, levomethadyl, spafloxacin, thioridazine, cisapride.

In certain embodiments, the arrhythmia comprises atrial fibrillation. In certain embodiments, the arrhythmia comprises atrial fibrillation, and the antiarrhythmic drug comprises amiodarone. In certain embodiments, the releasing member comprises a biodegradable material.

Certain embodiments provide a device, for treating or reducing a risk of postsurgical cardiac arrhythmia, comprising: a releasing member configured to be placed from outside a mammal's body, through an opening in the body, and into the mammal, and configured to be positioned between a visceral layer and a parietal layer of pericardium of the mammal's heart. In certain embodiments, the releasing member (i) releases an antiarrhythmic drug into the mammal's pericardial space, and (ii) is configured not to impede significantly a systolic or a diastolic function of the heart while the releasing member resides in the pericardial space. In certain embodiments, the releasing member releases the antiarrhythmic drug for a period of from about one day to about four days after the positioning. In certain embodiments, the device further comprises the antiarrhythmic drug. In some embodiments, the antiarrhythmic drug comprises amiodarone.

In certain embodiments, the antiarrhythmic drug comprises at least one of quinidine, procainamide, disopyramide, lidocaine, phenytoin, mexiletine, flecainide, propafenone, moricizine, propranolol, esmolol, timolol, metoprolol, atenolol, amiodarone, sotalol, ibutilide, dofetilide, verapamil, diltiazem, adenosine, digoxin, amiodarone, dofetilide, sotalol droperidol, levomethadyl, spafloxacin, thioridazine, cisapride.

In certain embodiments, the arrhythmia comprises atrial fibrillation. In certain embodiments, the arrhythmia comprises atrial fibrillation, and the antiarrhythmic drug comprises amiodarone. In certain embodiments, the releasing member comprises a biodegradable material.

Additional features and advantages of the invention will be set forth in the description below, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide further understanding of the invention and are incorporated in and constitute a part of this specification, illustrate aspects of the invention and together with the description serve to explain the principles of the invention.

FIG. 1 illustrates an example of a pericardially implantable member, in accordance with various embodiments of the subject disclosure.

FIG. 2 illustrates an example of a pericardially implantable member, in accordance with various embodiments of the subject disclosure.

FIG. 3 illustrates a method of treating or reducing a risk of postoperative cardiac arrhythmia, in accordance with various embodiments of the subject disclosure.

### DETAILED DESCRIPTION

In the following detailed description, numerous specific details are set forth to provide a full understanding of the present invention. It will be apparent, however, to one ordinarily skilled in the art that the present invention may be practiced without some of these specific details. In other instances, well-known structures and techniques have not been shown in detail so as not to obscure the present invention.

Coronary artery bypass (CAB) is performed on, e.g., patients with obstructive coronary artery disease to bypass blockages or obstructions of the coronary arteries and/or to repair or replace cardiac or pulmonary tissue in need thereof an. The high incidence of coronary artery disease, together with the effectiveness of CAB surgery, makes CAB surgery one of the top ten most frequently performed procedures in North America and Europe. According to the European Heart Survey and the National Registries of Cardiovascular Diseases and Patient Management, the total volume of bypass surgery was over 280,000 in the 15 European Union countries in the year 2000. In the United States it is estimated that over 700,000 CAB procedures are performed per year.

Despite the benefits of CAB surgery, patients undergoing these procedures may also suffer adverse outcomes including mortality, myocardial infarction, unstable angina, ventricular failure, arrhythmia, renal insufficiency, and stroke. Some of the proposed causes of cardiovascular morbidity and mortality after CAB include perioperative ischemia, inadequate myocardial protection, and reperfusion injury. The impact of these serious complications is significant. Estimates of incidence of death and myocardial infarction following CAB surgery range from 5% to 12%. Results from large clinical trials have demonstrated the importance of neurologic deficits as a problematic outcome of CAB. These deficits include impairment of memory, psychomotor, visuospatial, attention, and language abilities as measured by neuropsychological testing as well as sensorimotor abnormalities associated with stroke.

As used herein, the terms “cardiac procedure” and “cardiopulmonary bypass surgery” (CPB) refer to surgical procedures involving circulatory bypass of the heart and/or the lungs. CPB is often used in: surgery to repair blocked or clogged coronary arteries; surgery to repair or replace heart valves; surgery to correct cardiac arrhythmias, such as surgery to ablate (e.g., by thermal, microwave radiation, radio frequency radiation, etc. ablation) segments of heart tissue that create “short circuits” and heartbeat irregularities; surgery to remove heart muscle tissue to increase contact between a ventricular wall and oxygenated blood; heart transplant surgery; lung transplant surgery; and surgery to correct congenital heart disease.

During CPB surgery, the heart is usually stopped from beating while the heart and/or the lungs are being worked on so that surgeons do not perform delicate surgery on a moving target. The heartbeat is stopped by the combined effects of: perfusing the heart with a liquid (e.g., a cardioplegia solution) comprising a quantity of potassium sufficient to interfere with cellular electrochemical activities involved in initiation and control of heart contraction; chilling the heart; and clamping the aorta shut such that the left ventricle cannot pump blood. While the heartbeat is stopped, most of the patient's body (excluding the heart and, to some extent, the lungs) are supported by a bypass machine (also called a heart-lung machine) that receives deoxygenated blood from the patient's body, oxygenates the received blood, and pumps the oxygenated blood back into the patient's body. During CPB, the patient's brain and body are cooled several degrees, reducing consumption of oxygen by cells of the patient's brain body. Typically, the patient's heart must be chilled to a temperature that is substantially colder than the chilled temperature of patient's brain or body during CPB because the heart receives no oxygenated blood from the bypass machine during the bypass procedure.

After the surgical work requiring bypass is completed, surgeons flush the potassium liquid from the heart, and rewarm the heart muscle by passing warm blood or other liquid through the coronary arteries and veins. As the heart warms up, it usually begins to fibrillate, and surgeons use electrodes to defibrillate the heart, thereby restarting the heartbeat. When surgeons restart the heartbeat after bypass, ischemic damage to the heart can manifest in a variety of ways. In almost all CPB operations, at least some aberrations (including cardiac arrhythmias, abnormally rapid heart beat, abnormally slow heartbeat, ventricular fibrillation, or diminished pumping capacity) arise in varying degrees.

These aberrations in heart performance, triggered by ischemic and surgical insults experienced by the heart during the CPB operation, can result in a variety of health complications in patients following CPB surgery. For instance, the incidence of postcardiopulmonary AF in patients has been estimated at 20-35%. Since the atrial chambers are not contracting in AF, stasis of blood in the atrial chambers can lead to a blood clot within the heart that can then migrate to the brain, and result in a stroke. For this reason, many doctors prescribe, in response to an AF occurrence, blood thinners such as heparin or coumadin until the rhythm is returned to normal. In addition, many doctors prescribe antiarrhythmic medications in an effort to achieve a normal heartbeat rhythm. The overall result is that when postcardiopulmonary AF occurs, the patient's heart function declines, a risk of stroke and bleeding arises for the patient from the administration of blood thinners, side effects from the antiarrhythmic medications given to correct the AF can occur in the patient, a possibility that the antiarrhythmic medications will not work, leading to the need for electric shock to the patient's chest to correct the AF, etc. A consequence being that postcardiopulmonary AF can lead to an estimated 1.5 days in the hospital and at least an additional $10,000 of medical expenses.

As used herein, postcardiopulmonary procedure cardiac arrhythmia in a mammal comprises a cardiac arrhythmia occurrence within several days, such as one day, two days, three days, four days, five days, six days, or seven days, of the mammal undergoing a cardiopulmonary procedure. As used herein, postcardiopulmonary cardiac arrhythmia includes postcardiopulmonary AF.

Antiarrhythmic agents are a group of pharmaceuticals used to suppress fast heartbeat rhythms such as those resulting from atrial fibrillation, atrial flutter, ventricular tachycardia, and ventricular fibrillation. The Vaughan Williams (VW) antiarrhythmic agent classification scheme is widely used for antiarrhythmic agents, and it classifies a drug based on one of the following five mechanisms of action that produce its antiarrhythmic effect. There are five main classes in the Vaughan Williams classification of antiarrhythmic agents: class I agents interfere with the sodium (Na+) channel; class II agents are anti-sympathetic nervous system agents (e.g., beta blockers); class III agents affect potassium (K+) efflux; class IV agents affect calcium channels and the AV node; and class V agents work by other or unknown mechanisms.

Many antiarrhythmic agents have multiple mechanisms of action. Amiodarone, for example, has effects consistent with all of the VW classes I-IV. Another limitation is a lack of consideration within the VW classification system for the mechanisms of actions for drug metabolites. One such example is procainamide, a VW class I agent whose metabolite N-acetyl procainamide (NAPA) has a VW class III action.

Examples of class I antiarrhythmic agents include quinidine, procainamide, disopyramide, lidocaine, phenytoin, mexiletine, flecainide, propafenone, and moricizine. Clinical uses of VW class I antiarrhythmic agents include: treatment of ventricular arrhythmias and recurrent tachyarrhythmias of abnormal conduction systems; inhibition of paroxysmal recurrent AF; and treatment and inhibition of ventricular tachycardia and AF during and immediately after myocardial infarction, though this practice has been discouraged given the increased risk of asystole.

Examples of class II antiarrhythmic agents include propranolol, esmolol, timolol, metoprolol, and atenolol. Clinical uses of class II antiarrhythmic agents include treatment of myocardial infarction and inhibition of recurrences of tachyarrhythmias.

Examples of class III antiarrhythmic agents include amiodarone, sotalol, ibutilide, and dofetilide. Clinical uses of class III antiarrhythmic agents include treatment of Wolff-Parkinson-White syndrome, ventricular tachycardias, and AF.

Examples of class IV antiarrhythmic agents include verapamil and diltiazem. Clinical uses of VW class IV antiarrhythmic agents include treatment of AF and inhibition of recurrence of paroxysmal supraventricular tachycardia.

Examples of class V antiarrhythmic agents include adenosine and digoxin. Clinical uses of class V antiarrhythmic agents include treatment of reduction of the conduction of electrical impulses through the AV node.

In certain embodiments, a regimen of antiarrhythmic drug exposure to cardiac tissue of a mammal that has undergone a cardiopulmonary procedure comprises a frequency, duration, and amount of antiarrhythmic drug exposure to a cardiac tissue. As used herein, an antiarrhythmic drug can comprise an antiarrhythmic drug and/or an antiarrhythmic agent.

Additional antiarrhythmic drugs useful in certain embodiments of the subject disclosure include amiodarone, dofetilide, sotalol, droperidol, levomethadyl, spafloxacin, thioridazine, cisapride.

Antiarrhythmic drugs produce a group of side effects related to excessive dosage and plasma concentration that are in both noncardiac (e.g., neurological symptoms) and cardiac (e.g., dysrhythmias) toxicity. Examples of noncardiac toxicity include tinnitus, hearing loss, visual disturbances, confusion, delirium, allergic reactions, and psychosis. Examples of cardiac toxicity include ventricular tachycardia, reduced ventricular function, atrial tachycardia, and reduced atrial function.

Antiarrhythmic drugs produce another group of side effects, unrelated to plasma concentrations, termed idiopathic, which include procainamide-induced lupus syndrome, amiodarone-induced pulmonary toxicity, and quinidine-induced torsades de pointes.

In certain embodiments, a duration of exposure of cardiac tissue to an antiarrhythmic drug released by a source of the antiarrhythmic drug can range from about one hour to about seven days. In certain embodiments the duration of release by an antiarrhythmic drug source can be about one hour, about two hours, about three hours, about four hours, about five hours, about six hours, about 12 hours, about one day, about two days, about three days, about four days, about five days, about six days, and about seven days. In certain embodiments, a frequency of antiarrhythmic drug release by a source of an antiarrhythmic drug can be continuously throughout the duration of release.

In certain embodiments, a duration of exposure of cardiac tissue to an antiarrhythmic drug used by the source of the antiarrhythmic drug can begin at a time later than the time at which the source is initially positioned at least partially within the pericardium of the patient. A source of an antiarrhythmic drug can be configured so that an antiarrhythmic drug release delay time from the source following its initial positioning at least partially within the pericardium of the patient is about one minute, about two minutes, about three minutes, about four minutes, about five minutes, about 10 minutes, about 15 minutes, about 30 minutes, about 45 minutes, about one hour, about two hours, about three hours, about four hours, about five hours, about six hours, about seven hours, about eight hours, about nine hours, about 10 hours, about 11 hours, and about 12 hours.

In certain embodiments, a frequency of exposure of cardiac tissue to an antiarrhythmic drug released by a source of the antiarrhythmic drug can comprise a percentage of time of the release duration, such as about 1%, about 2%, about 5%, about 10%, about 20%, about 30%, about 40%, about 50%, about 60%, about 70%, about 80%, about 90%, and about 95% of the release duration, the percentage of time of the release duration substantially uniformly distributed throughout the duration of release in intervals with the percentage of time of non-release of the antiarrhythmic drug.

In certain embodiments, an amount of an antiarrhythmic drug to which cardiac tissue is exposed by release of the antiarrhythmic drug from the source include those effective to result in a pericardial space concentration of the antiarrhythmic drug in a range of from about 1 nanomolar to about 1 molar. In certain embodiments, such a concentration of antiarrhythmic drug can be about 1 nanomolar, about 10 nanomolar, about 100 nanomolar, about 1 micromolar, about 10 micromolar, about 100 micromolar, about 1 millimolar, about 10 millimolar, about 100 millimolar, and about 1 molar.

In certain embodiments, a source of an antiarrhythmic drug can comprise an amount of the antiarrhythmic drug in a range of from about 1 nanogram to about 10 grams. In certain embodiments, such amounts of antiarrhythmic drug can be about 1 nanogram, about 10 nanograms, about 100 nanograms, about 1 milligram, about 10 milligrams, about 100 milligrams, about 1 gram, about 2.5 grams, about 5 grams, about 7.5 grams, and about 10 grams.

Preparations of an antiarrhythmic drug source useful in certain embodiments can comprise slow-release biocompatible and/or biodegradable formulations and mechanisms comprising polymers, such as polylactide polymers, polyglycolide polymers, polylactide-polyglycolide copolymers, hyaluronic acid, carboxycellulose, amylose, and mixtures thereof. Many such slow-release formulations and mechanisms are known and described in, for instance, U.S. Pat. No. 5,641,745, USPAP 2008/0249633, and U.S. Pat. No. 6,074,673, the contents of each of which are hereby incorporated by reference in their entireties. As used herein, the term “biodegradable” can refer to polymers and other compositions that are degradable in vivo, either enzymatically or non-enzymatically, to produce biocompatible or non-toxic by-products which can be further metabolized or excreted via normal physiological pathways. A biodegradable material can comprise a biologically-compatible, self-absorbing matrix. Examples of synthetic biodegradable polymers include poly(lactide); poly(glycolide) and poly(lactide-co-glycolide), steroisomers (i.e., D, L), racemic mixtures, and polymer mixtures thereof.

Delivery methods for a source of an antiarrhythmic drug to a position at least partially within the target of a cardiopulmonary procedure patient include surgical implantation, such as during open chest surgery or such as by catheter.

In certain embodiments, a source of an antiarrhythmic drug can comprise a plurality of antiarrhythmic drugs. In certain embodiments, a source of antiarrhythmic drug can comprise one or more antiarrhythmic drugs such and one or more additional bioactive substances, such as an anticoagulant (blood “thinning”) compound (e.g., heparin and coumadin) and an antibiotic (e.g. penicillin, cephalosporin, polymixin, quinolone, sulfonamide, aminoglycoside, macrolide and tetracycline). In certain embodiments, a antiarrhythmic drug source can comprise an additional bioactive substance in an amount in a range of from about 1 nanogram to about 10 grams. In certain embodiments, such amounts of antiarrhythmic drug can be about 1 nanogram, about 10 nanograms, about 100 nanograms, about 1 milligram, about 10 milligrams, about 100 milligrams, about 1 gram, about 2.5 grams, about 5 grams, about 7.5 grams, and about 10 grams.

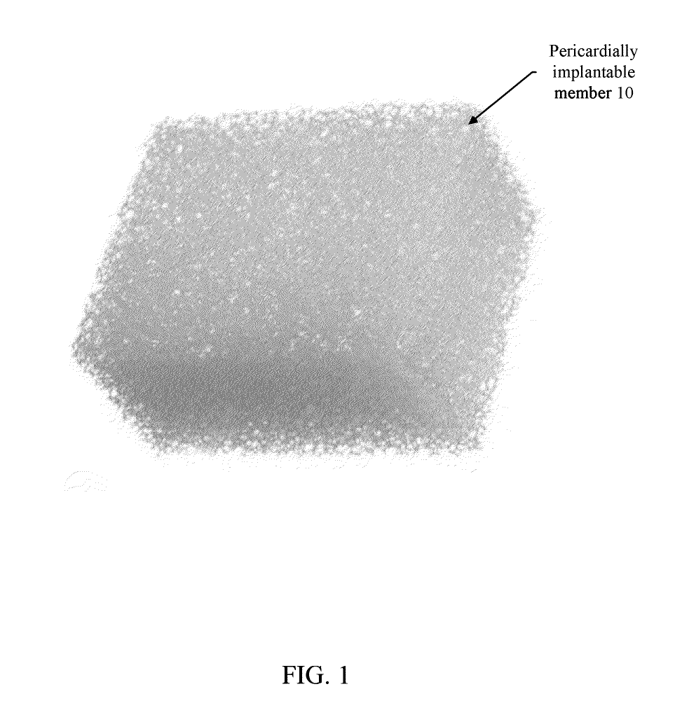

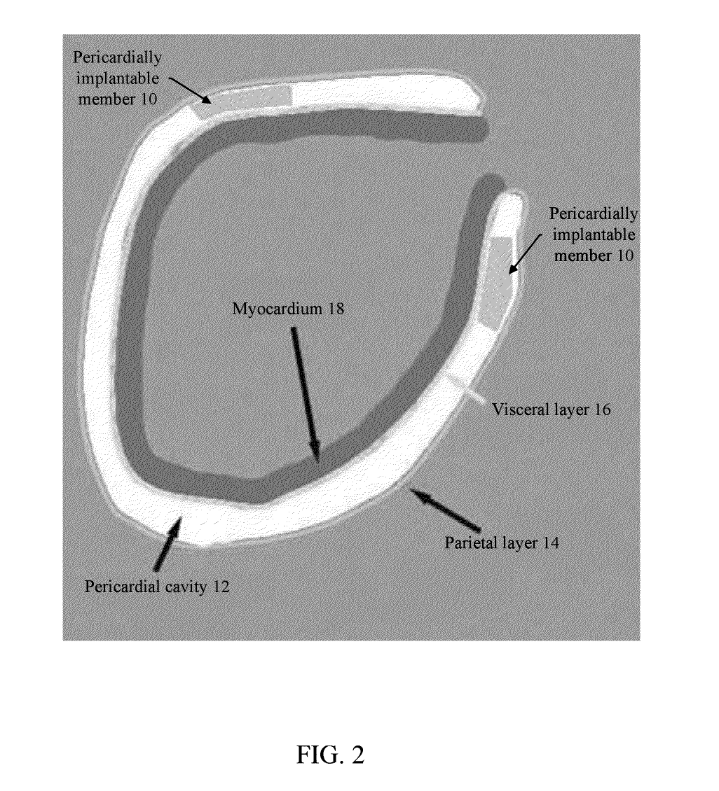

A source of an antiarrhythmic drug useful in certain embodiments of the subject disclosure comprises a pericardially implantable member reversibly coupled to an antiarrhythmic drug. FIGS. 1 and 2 illustrate an example of a pericardially implantable member 10, in accordance with various embodiments of the subject disclosure. A mammal's heart may comprise a myocardium 18 and a pericardium. The pericardium may comprise a visceral layer 16 and a parietal layer 14. The pericardial cavity 12 may be the space between the visceral layer 16 and the parietal layer 14. In some embodiments, the pericardially implantable member 10 may be disposed between the visceral layer 16 and the parietal layer 14. In certain embodiments, the pericardially implantable member 10 comprises materials conferring slow release antiarrhythmic drug properties. In certain embodiments, the pericardially implantable member 10 comprises a biologically compatible and/or biodegradable material reversibly coupled to the antiarrhythmic drug. In certain embodiments, materials conferring slow release antiarrhythmic drug properties comprise polymers and/or porous membranes. In certain embodiments, the form of a source of an antiarrhythmic drug can comprise a pad, a bag, a film, a patch, a sponge, etc., or other suitable forms known to those of skill in the art. In certain embodiments, a source of an antiarrhythmic drug can comprise a gel, a paste, etc.

In some embodiments, an average thickness of the pericardially implantable member 10 is less than or equal to about 20 millimeters (mm). In some embodiments, an average thickness of the pericardially implantable member 10 is less than or equal to about 15 mm. In some embodiments, an average thickness of the pericardially implantable member 10 is less than or equal to about 10 mm. In some embodiments, an average thickness of the pericardially implantable member 10 is less than or equal to about 5 mm. In some embodiments, an average thickness of the pericardially implantable member 10 is less than or equal to about 3 mm. In some embodiments, an average thickness of the pericardially implantable member 10 is less than or equal to about 2 mm. In some embodiments, an average thickness of the pericardially implantable member 10 is less than or equal to about 1 mm. In some embodiments, the pericardially implantable member 10 can include adhesive, glue, one or more anchor members, or other suitable mechanisms to hold the pericardially implantable member 10 in place in the pericardial cavity 12.

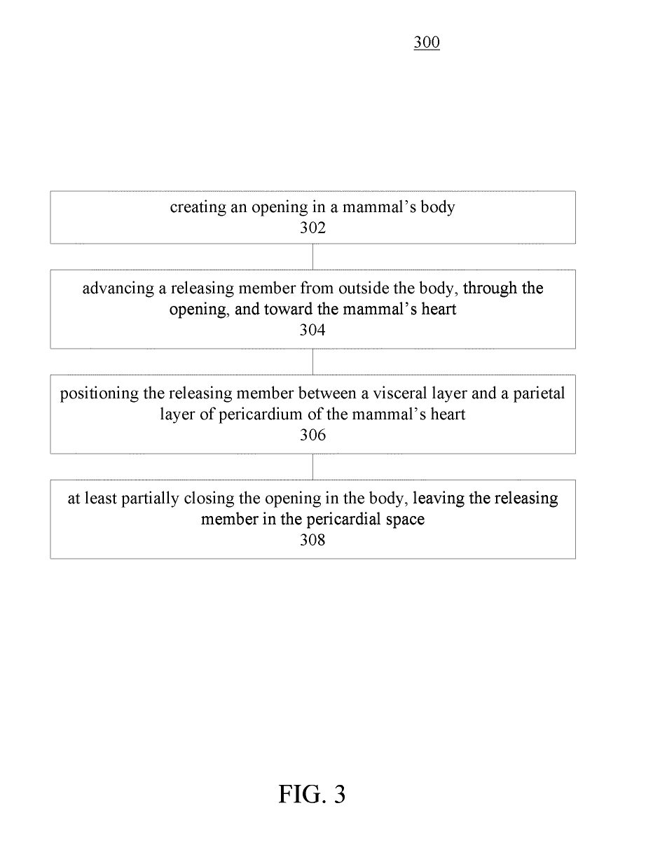

FIG. 3 illustrates a method 300 of treating or reducing a risk of postoperative cardiac arrhythmia, in accordance with various embodiments of the subject disclosure. Method 300 may comprise: creating an opening in a mammal's body (302); advancing a releasing member from outside the body, through the opening, and toward the mammal's heart (304); positioning the releasing member between a visceral layer and a parietal layer of pericardium of the mammal's heart (306); and at least partially closing the opening in the body, leaving the releasing member in the pericardial space (308). In certain embodiments, the releasing member releases an antiarrhythmic drug into the mammal's pericardial space and is configured not to impede significantly a systolic or a diastolic function of the heart while the releasing member resides in the pericardial space.

The foregoing description is provided to enable a person skilled in the art to practice the various configurations described herein. While the present invention has been particularly described with reference to the various figures and configurations, it should be understood that these are for illustration purposes only and should not be taken as limiting the scope of the invention.

There may be many other ways to implement the invention. Various functions and elements described herein may be partitioned differently from those shown without departing from the scope of the invention. Various modifications to these configurations will be readily apparent to those skilled in the art, and generic principles defined herein may be applied to other configurations. Thus, many changes and modifications may be made to the invention, by one having ordinary skill in the art, without departing from the scope of the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

What is claimed is:

1. A method, of treating or reducing a risk of postoperative cardiac arrhythmia, comprising:
creating an opening in a mammal's body;
advancing a releasing member from outside the body, through the opening, and toward the mammal's heart;
positioning the releasing member between a visceral layer and a parietal layer of pericardium of the mammal's heart; and
at least partially closing the opening in the body, leaving the releasing member in the pericardial space;
wherein the releasing member releases an antiarrhythmic drug into the mammal's pericardial space and is configured not to impede significantly a systolic or a diastolic function of the heart while the releasing member resides in the pericardial space.

2. The method of claim 1, wherein the positioning occurs before or after a cardiopulmonary surgery on the heart that is performed before the at least partial closing.

3. The method of claim 2, wherein the cardiopulmonary procedure comprises at least one surgery selected from surgery to bypass or repair stenotic coronary arteries; surgery for a heart valve; surgery to correct a cardiac arrhythmia; surgery to remove heart muscle tissue; heart transplant surgery; lung surgery; and surgery to correct a congenital heart disease.

4. The method of claim 1, wherein the antiarrhythmic drug comprises at least one of quinidine, procainamide, disopyramide, lidocaine, phenytoin, mexiletine, flecainide, propafenone, moricizine, propranolol, esmolol, timolol, metoprolol, atenolol, amiodarone, sotalol, ibutilide, dofetilide, verapamil, diltiazem, adenosine, digoxin, amiodarone, dofetilide, sotalol droperidol, levomethadyl, spafloxacin, thioridazine, cisapride.

5. The method of claim 1, wherein the arrhythmia comprises atrial fibrillation.

6. The method of claim 1, wherein the arrhythmia comprises atrial fibrillation, and the antiarrhythmic drug comprises amiodarone.

7. The method of claim 1, wherein the releasing member comprises a biodegradable material.

8. The method of claim 1, wherein the releasing member releases the antiarrhythmic drug for a period of from about one day to about four days after the positioning.

9. The method of claim 1, further comprising permitting at least a portion of the releasing member to biodegrade in the mammal's body.

10. A device, for treating or reducing a risk of postsurgical cardiac arrhythmia, comprising:
a releasing member configured to be placed from outside a mammal's body, through an opening in the body, and into the mammal, and configured to be positioned between a visceral layer and a parietal layer of pericardium of the mammal's heart;
wherein the releasing member (i) releases an antiarrhythmic drug into the mammal's pericardial space, and (ii) is configured not to impede significantly a systolic or a diastolic function of the heart while the releasing member resides in the pericardial space;
wherein the releasing member at least partially resorbs in the mammal's body over at least two days following placement in the pericardial space.

11. The device of claim 10, further comprising the antiarrhythmic drug, the antiarrhythmic drug comprising amiodarone.

12. The device of claim 10, further comprising the antiarrhythmic drug, the antiarrhythmic drug comprising at least one of quinidine, procainamide, disopyramide, lidocaine, phenytoin, mexiletine, flecainide, propafenone, moricizine, propranolol, esmolol, timolol, metoprolol, atenolol, amiodarone, sotalol, ibutilide, dofetilide, verapamil, diltiazem, adenosine, digoxin, amiodarone, dofetilide, sotalol droperidol, levomethadyl, spafloxacin, thioridazine, cisapride.

13. The device of claim 10, wherein the arrhythmia comprises atrial fibrillation.

14. The device of claim 10, wherein the arrhythmia comprises atrial fibrillation, and the antiarrhythmic drug comprises amiodarone.

15. The device of claim 10, wherein the releasing member comprises a biodegradable material.

16. A device, for treating or reducing a risk of postsurgical cardiac arrhythmia, comprising:
a releasing member configured to be placed from outside a mammal's body, through an opening in the body, and into the mammal, and configured to be positioned between a visceral layer and a parietal layer of pericardium of the mammal's heart;
wherein the releasing member (i) releases an antiarrhythmic drug into the mammal's pericardial space, and (ii) is configured not to impede significantly a systolic or a diastolic function of the heart while the releasing member resides in the pericardial space;
wherein the releasing member releases the antiarrhythmic drug for a period of from about one day to about four days after the positioning.

17. The device of claim 16, further comprising the antiarrhythmic drug, the antiarrhythmic drug comprising amiodarone.

18. The device of claim 16, further comprising the antiarrhythmic drug, the antiarrhythmic drug comprising at least one of quinidine, procainamide, disopyramide, lidocaine, phenytoin, mexiletine, flecainide, propafenone, moricizine, propranolol, esmolol, timolol, metoprolol, atenolol, amiodarone, sotalol, ibutilide, dofetilide, verapamil, diltiazem, adenosine, digoxin, amiodarone, dofetilide, sotalol droperidol, levomethadyl, spafloxacin, thioridazine, cisapride.

19. The device of claim 16, wherein the arrhythmia comprises atrial fibrillation, and the antiarrhythmic drug comprises amiodarone.

20. The device of claim 16, wherein the releasing member comprises a biodegradable material.
